(19) United States
(12) Patent Application Publication
(10) Pub. No.: US 2025/0383346 A1
(43) Pub. Date: Dec. 18, 2025

(54) AFFINITY SCREENING METHOD

(71) Applicant: ABSCI CORPORATION, Vancouver, WA (US)

(72) Inventors: Sharrol Bachas, Vancouver, WA (US); Robel Haile, Vancouver, WA (US); Engin Yapici, Vancouver, WA (US); Matthew Weinstock, Camas, WA (US)

(21) Appl. No.: 18/878,245

(22) PCT Filed: Jun. 27, 2023
(86) PCT No.: PCT/US2023/026332

§ 371 Date: Dec. 23, 2024

Related U.S. Application Data
(60) Provisional application No. 63/356,801, filed on Jun. 29, 2022.

(51) Int. Cl. G01N 33/543; G01N 33/68

(52) U.S. Cl. CPC G01N 33/543; G01N 33/6854

(57) ABSTRACT

Described herein is a method of determining affinity of an antigen binding protein binding to a target, the method comprising: a) expressing an antigen binding protein in a prokaryotic host cell in a multi-well container, b) lysing the prokaryotic cell to produce a prokaryotic cell lysate; c) affixing the prokaryotic cell lysate to a substrate; and d) determining the affinity of the antigen binding protein to the target.

### BACKGROUND

Antibody production in prokaryotic systems is challenging. Traditionally, researchers relied on eukaryotic production and purification to study the antigen-binding properties of antibodies of interest. The overall process of antibody production and antigen binding analysis is tedious, costly and time consuming. These limitations hamper protein engineering efforts for designing and screening novel biotherapeutics. Thus, there remains a need in the art for high-throughput methods for determining antibody affinity.

### SUMMARY

In one aspect, described herein is a method of determining affinity of an antigen binding protein binding to a target, the method comprising: a) expressing an antigen binding protein in a prokaryotic host cell in a multi-well container, b) lysing the prokaryotic cell to produce a prokaryotic cell lysate; c) affixing the prokaryotic cell lysate to a substrate; and d) determining the affinity of the antigen binding protein to the target.

In another aspect, described herein is a method of determining affinity of a multi-chain protein binding to a target, the method comprising: a) expressing a multi-chain protein in a prokaryotic host cell, b) lysing the prokaryotic cell to produce a prokaryotic cell lysate; c) affixing the prokaryotic cell lysate to a substrate; and d) determining the affinity of the multi-chain protein to the target.

In some embodiments, wherein the prokaryotic host cell comprises (a) an alteration of gene function of at least one gene encoding a transporter protein for an inducer of at least one inducible promoter; (b) a reduced level of gene function of at least one gene encoding a protein that metabolizes an inducer of at least one inducible promoter; (c) a reduced level of gene function of at least one gene encoding a protein involved in biosynthesis of an inducer of at least one inducible promoter; (d) an altered gene function of a gene that affects the reduction/oxidation environment of the host cell cytoplasm; (e) a reduced level of gene function of a gene that encodes a reductase; (f) at least one expression construct encoding at least one disulfide bond isomerase protein; (g) at least one polynucleotide encoding a form of DsbC lacking a signal peptide; and/or (h) at least one polynucleotide encoding Ervlp.

### BRIEF DESCRIPTION OF FIGURES

FIG. 1 provides a schematic of methods for direct antigen binding analysis of antibodies in bacterial production cell lines. Workflow showing a capture example of Trastuzumab Fab antibody fragment from lysate followed by direct analysis of Her2 binding using high-throughput SPR methods.

FIGS. 2A-2D demonstrate superior antibody capture performance from crude lysate using methods described in the Examples as compared to an alternative capture methods. FIG. 2A provides depictions of alternative capture method using C-tag capture of antibody Fab fragment on Carterra chip surface. FIG. 2B provides depictions of alternative capture method using Fab kappa capture on Carterra chip surface. FIG. 2C provides an immobilization sensorgram of C-tag capture showing time-dependent dissociation (slated lines) of captured antibodies a baseline step prior to antigen binding. FIG. 2D provides immobilization sensorgram showing efficient capture of antibody Fab fragment using Fab Kappa capture method. At baseline washout step lines are flat indicating stable capture of Fabs and no time-dependent dissociation prior to antibody binding step.

FIG. 3A shows the comparison of the accuracy of SPR lysate binding data to purified antibodies by an orthogonal technique. Variants of Trastuzumab in lysate were analyzed for Her2 binding on SPR and kinetics compared to binding of purified variants by Biolayer Interferometry (BLI). Pearson coefficient of 0.95 shows good correlation between KD from lysate SPR and purified BLI values.

FIG. 3B shows the comparison of the accuracy of SPR lysate binding data to purified SPR binding data. Variants of Trastuzumab in lysate were analyzed for Her2 binding on SPR and kinetics compared to binding of purified variants by SPR.

FIGS. 4A and 4B show the validation of the sensitivity and limit of detection of antibody binding kinetic based on methods described in the Examples. FIG. 4A shows dissociation constant (KD) vs. concentration of Trastuzumab Mab or Fab in lysate or purified formats. FIG. 4B shows the maximum binding response, Rmax vs. concentration of Trastuzumab Mab or Fab in lysate or purified formats.

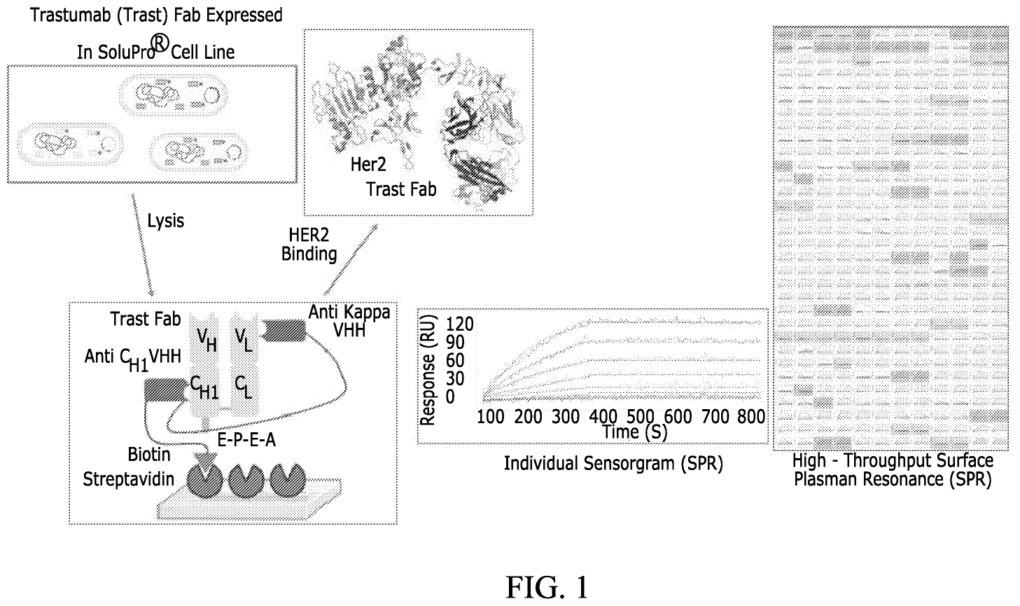

### DETAILED DESCRIPTION

The present disclosure is based on the development of a rapid and high-throughput method for analyzing the antigen-binding properties of antigen binding proteins (e.g., antibodies) directly from bacterial lysate. As described in the Examples, clarified bacterial lysate is analyzed by high throughput analysis (e.g., surface plasmon resonance (SPR)) to determine kinetic parameters of antibody binding, which allows for the direct analysis of antigen binding in optimized prokaryotic strains designed for antibody production.

In one aspect, described herein is a method of determining affinity of an antigen binding protein binding to a target, the method comprising: a) expressing an antigen binding protein in a prokaryotic host cell in a multi-well container, b) lysing the prokaryotic cell to produce a prokaryotic cell lysate; c) affixing the prokaryotic cell lysate to a substrate; and d) determining the affinity of the antigen binding protein to the target.

In another aspect, described herein is a method of determining affinity of an multi-chain protein binding to a target, the method comprising: a) expressing an antigen binding protein in a prokaryotic host cell in a multi-well container, b) lysing the prokaryotic cell to produce a prokaryotic cell lysate; c) affixing the prokaryotic cell lysate to a substrate; and d) determining the affinity of the multi-chain protein to the target.

In some embodiments, the prokaryotic cells are lysed, insoluble debris removed, and the soluble fraction is used in a binding assay without any additional purification steps or with minimal purification. In such aspects, the antigen binding protein (or multi-chain protein) is only a fraction of the protein content within the sample. In some embodiments, the antigen binding protein (or multi-chain protein) is at most 60%, at most 55%, at most 50%, at most 45%, at most 40%, at most 35%, at most 30%, at most 25%, at most 20%, at most 15% or at least 5%, at least 10%, at least 15%, at least 20%, at least 25%, at least 30%, at least 35%, at least 40%, at least 45%, or at least 50% of the protein content within the sample. The antigen binding protein or multi-chain protein may be from 5% to 60%, 10% to 40%, or 15% to 30% of the protein content within the sample.

Binding assays, for example assays that measure protein-protein interactions, including antibody-antigen interactions and including measuring binding affinity, are well known in the art. By way of example, Surface plasmon resonance (SPR), Bio-layer interferometry (BLI), Dual polarisation interferometry (DPI), Static light scattering (SLS), Dynamic light scattering (DLS), Flow-induced dispersion analysis (FIDA), Fluorescence polarization/anisotropy, Fluorescence resonance energy transfer (FRET), Isothermal titration calorimetry (ITC), Microscale thermophoresis (MST), Single colour reflectometry (SCORE) are contemplated. Additionally, Bimolecular fluorescence complementation (BiFC), affinity electrophoresis, label transfer, phage display, Tandem affinity purification (TAP), cross-linking, Quantitative immunoprecipitation combined with knock-down (QUICK) and Proximity ligation assay (PLA) are other well-known assays that provide protein-protein interaction information.

Antigen binding proteins can be captured on any substrate for use in the methods described herein. Exemplary substrates (e.g., optionally in association with a capture agent as described herein) include, but are not limited to, metal surfaces coated with one or more of the following: covalent conjugation to polycarboxylate hydrogel and caboxymethyldextran matrices, protein A/G derivatived hydrogel and caboxymethyldextran matrices, Streptavidin derivatized hydrogel and caboxymethyldextran. Exemplary capture agents include, but are not limited to, kappa capture, lambda capture, FLAG tag capture, HIS tag capture, Protein A, Protein G, Protein L, and biotin/streptavidin.

In some embodiments, the binding affinities of antigen binding proteins are measured by array surface plasmon resonance (SPR), according to standard techniques (Abdiche, et al. (2016) MAbs 8:264-277). Briefly, antibodies were immobilized on a HC 30M chip at four different densities/antibody concentrations. Varying concentrations (0-500 nM) of antibody target are then bound to the captured antibodies. Kinetic analysis is performed using Carterra software to extract association and dissociation rate constants (kₐ and k_{d}, respectively) for each antibody. Apparent affinity constants (K_{D}) are calculated from the ratio of k_{d}/kₐ. In some embodiments, the Carterra LSA Platform is used to determine kinetics and affinity. In other embodiments, binding affinity can be measured, e.g., by surface plasmon resonance (e.g., BIAcore™) using, for example, the IBIS MX96 SPR system from IBIS Technologies or the Carterra LSA SPR platform, or by Bio-Layer Interferometry, for example using the Octet™ system from ForteBio. In some embodiments, a biosensor instrument such as Octet RED384, ProteOn XPR36, IBIS MX96 and Biacore T100 is used (Yang, D., et al., J. Vis. Exp., 2017, 122:55659).

K_{D} is the equilibrium dissociation constant, a ratio of K_{off}/kₒₙ, between the antigen binding protein (e.g., antibody) and its antigen. K_{D} and affinity are inversely related. The K_{D} value relates to the concentration of antibody and so the lower the K_{D} value (lower concentration) and thus the higher the affinity of the antibody. Antibody, including reference antibody and variant antibody, K_{D} according to various embodiments of the present disclosure can be, for example, in the micromolar range (10⁻⁴ to 10⁻⁶), the nanomolar range (10⁻⁷ to 10⁻⁹), the picomolar range (10⁻¹⁰ to 10⁻¹²) or the femtomolar range (10⁻¹³ to 10⁻¹⁵). In some embodiments, antibody affinity of a variant antibody is improved, relative to a reference antibody, by approximately 5, 10, 15, 20, 25, 30, 35, 40, 45, or 50% or more. The improvement may also be expressed relative to a fold change (e.g., 2×, 4×, 6×, or 2-, 3-, 4-, 5-, 6-, 7-, 8-, 9-, 10-fold or more improvement in binding activity, etc.) and/or an order of magnitude (e.g., 10⁷, 10⁸, 10⁹, etc.).

### Antigen Binding Proteins

Antigen binding proteins contemplated herein include full-length proteins, precursors of full-length proteins, biologically active subunits or fragments of full length proteins, as well as biologically active derivatives and variants of any of these forms of therapeutic proteins. Thus, antigen binding proteins include those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a polypeptide encoded by a referenced nucleic acid or an amino acid sequence described herein. According to the present disclosure, the term “recombinant protein” includes any protein obtained via recombinant DNA technology. In certain embodiments, the term encompasses proteins as described herein.

In some embodiments, the antigen binding protein is an antibody. The term “antibody” as used herein refers to whole antibodies that interact with (e.g., by binding, steric hindrance, stabilizing/destabilizing, spatial distribution) an epitope on a target antigen.

The term “multi-chain protein” as used herein refers to an antibody comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, single-chain Fvs (scFv), disulfide-linked Fvs (sdFv), Fab fragments, F(ab′) fragments, and anti-idiotypic (anti-Id) antibodies (including, e.g., anti-Id antibodies to antibodies of the invention), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass. The antibody or epitope-binding fragments may be, or be a component of, a multi-specific molecule.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

Exemplary antibodies include, but are not limited to, antibodies such as infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, and zolimomab aritox.

The phrase “antibody fragment”, as used herein, refers to one or more portions of an antibody that retain the ability to specifically interact with (e.g., by binding, steric hindrance, stabilizing/destabilizing, spatial distribution) a target epitope. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies.

### Prokaryotic Cells

The methods described herein comprise expressing an antigen binding protein (or a multi-chain binding protein) in a prokaryotic host cell. Prokaryotic cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

As described in International Publication No. WO 2017/106583, incorporated by reference in its entirety herein, producing an antigen binding protein at commercial scale and in soluble form is addressed by providing suitable host cells capable of growth at high cell density in fermentation culture, and which can produce soluble gene products in the oxidizing host cell cytoplasm through highly controlled inducible gene expression. Prokaryotic cells with these qualities are produced by combining some or all of the following characteristics: (1) The host cells are genetically modified to have an oxidizing cytoplasm, through increasing the expression or function of oxidizing polypeptides in the cytoplasm, and/or by decreasing the expression or function of reducing polypeptides in the cytoplasm. Specific examples of such genetic alterations are provided herein. Optionally, host cells can also be genetically modified to express chaperones and/or cofactors that assist in the production of the desired gene product(s), and/or to glycosylate polypeptide gene products. (2) The host cells comprise one or more expression constructs designed for the expression of one or more gene products of interest; in certain embodiments, at least one expression construct comprises an inducible promoter and a polynucleotide encoding a gene product to be expressed from the inducible promoter. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araE, araG, araH, rhaT, xyIF, xyIG, and xyIH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is selected from the group consisting of araA, araB, araD, prpB, prpD, rhaA, rhaB, rhaD, xylA, and xyIB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmIA, rmIB, rmIC, and rmlD.

Prokaryotic Cells with Oxidizing Cytoplasm. Examples of host cells are provided that allow for the efficient and cost-effective expression of gene products, including components of multimeric products. Host cells can include, in addition to isolated cells in culture, cells that are part of a multicellular organism, or cells grown within a different organism or system of organisms. In certain embodiments of the disclosure, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli (B strain) EB0001 cells (also called E. coli ASE (DGH) cells), or are E. coli (B strain) EB0002 cells. In growth experiments with E. coli host cells having oxidizing cytoplasm, specifically the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H), these E. coli B strains with oxidizing cytoplasm are able to grow to much higher cell densities than the most closely corresponding E. coli K strain (International Publication No. WO 2017/106583).

Certain alterations can be made to the gene functions of host cells comprising inducible expression constructs, to promote efficient and homogeneous induction of the host cell population by an inducer. In some embodiments, the combination of expression constructs, host cell genotype, and induction conditions results in at least 75% (more preferably at least 85%, and most preferably, at least 95%) of the cells in the culture expressing gene product from each induced promoter, as measured by the method of Khlebnikov et al. described in Example 9 of International Publication No. WO 2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the gene protein-coding sequence in its entirety, or deleting a large enough portion of the gene, inserting sequence into the gene, or otherwise altering the gene sequence so that a reduced level of functional gene product is made from that gene. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of chaperone proteins, it is also possible to alter the reduction-oxidation environment of the host cell.

Host cell reduction-oxidation environment. In bacterial cells such as E. coli, proteins that need disulfide bonds are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 2011 May 14; 10:32). It is also possible to express cytoplasmic forms of these Dsb proteins, such as a cytoplasmic version of DsbA and/or of DsbC (′cDsbA or ‘cDsbC’), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in heterologous proteins produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (such as ahpC* and ahpCA, Lobstein et al., Microb Cell Fact 2012 May 8; 11:56; doi: 10.1186/1475-2859-11-56) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dupl62-169 (Faulkner et al., Proc Natl Acad Sci USA 2008 May 6; 105 (18): 6735-6740, Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase I) catalase-peroxidase encoded by E. coli katG and HPII (hydroperoxidase II) catalase-peroxidase encoded by E. coli katE, which disproportionate peroxide into water and 02 (Farr and Kogoma, Microbiol Rev. 1991 December; 55 (4): 561-585; Review). Increasing levels of KatG and/or KatE protein in host cells through induced coexpression or through elevated levels of constitutive expression is an aspect of some embodiments of the disclosure.

Another alteration that can be made to host cells is to express the sulfhydryl oxidase Ervlp from the inner membrane space of yeast mitochondria in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 2011 Jan. 7; 10:1).

Host cells comprising expression constructs preferably also express cDsbA and/or cDsbC and/or Ervlp; are deficient in trxB gene function; are also deficient in the gene function of either gor, gshB, or gshA; optionally have increased levels of katG and/or katE gene function; and express an appropriate mutant form of AhpC so that the host cells can be grown in the absence of DTT.

Chaperones. In some embodiments, desired gene products are coexpressed with other gene products, such as chaperones, that are beneficial to the production of the desired gene product. Chaperones are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome; preferably, expression of chaperone protein(s) in the host cell is at a sufficiently high level to produce coexpressed gene products that are properly folded and/or assembled into the desired product. Examples of chaperones present in E. coli host cells are the folding factors DnaK/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. Dnak/DnaJ/GrpE, GroEL/GroES, and ClpB can function synergistically in assisting protein folding and therefore expression of these chaperones in combinations has been shown to be beneficial for protein expression (Makino et al., Microb Cell Fact 2011 May 14; 10:32). When expressing eukaryotic proteins in prokaryotic host cells, a eukaryotic chaperone protein, such as protein disulfide isomerase (PDI) from the same or a related eukaryotic species, is in certain embodiments of the disclosure coexpressed or inducibly coexpressed with the desired gene product.

One chaperone that can be expressed in host cells is a protein disulfide isomerase from Humicola insolens, a soil hyphomycete (soft-rot fungus). An amino acid sequence of Humicola insolens PDI is shown as SEQ ID NO: 1 of International Publication No. WO 2017/106583; it lacks the signal peptide of the native protein so that it remains in the host cell cytoplasm. The nucleotide sequence encoding PDI was optimized for expression in E. coli; the expression construct for PDI is shown as SEQ ID NO: 2 of International Publication No. WO 2017/106583. SEQ ID NO: 2 contains a GCTAGC Nhel restriction site at its 5′ end, an AGGAGG ribosome binding site at nucleotides 7 through 12, the PDI coding sequence at nucleotides 21 through 1478, and a GTCGAC Sail restriction site at its 3′ end. The nucleotide sequence of SEQ ID NO: 2 was designed to be inserted immediately downstream of a promoter, such as an inducible promoter. The Nhel and Sail restriction sites in SEQ ID NO: 2 can be used to insert it into a vector multiple cloning site, such as that of the pSOL expression vector (SEQ ID NO: 3 of International Publication No. WO 2017/106583), described in published US patent application US 2015/353940A1, which is incorporated by reference in its entirety herein. Other PDI polypeptides can also be expressed in host cells, including PDI polypeptides from a variety of species (Saccharomyces cerevisiae (UniProtKB PI 7967), Homo sapiens (UniProtKB P07237), Mus musculus (UniProtKB P09103), Caenorhabditis elegans (UniProtKB Q 17770 and Q 17967), Arabdopsis thaliana (UniProtKB 048773, Q9X101, Q9S G3, Q9LJU2, Q9MAU6, Q94F09, and Q9T042), Aspergillus niger (UniProtKB Q12730) and also modified forms of such PDI polypeptides. In certain embodiments of the disclosure, a PDI polypeptide expressed in host cells of the disclosure shares at least 70%, or 80%, or 90%, or 95% amino acid sequence identity across at least 50% (or at least 60%, or at least 70%, or at least 80%, or at least 90%) of the length of SEQ ID NO: 1 of International Publication No. WO 2017/106583, where amino acid sequence identity is determined according to Example 10 of International Publication No. WO 2017/106583.

Cellular transport of cofactors. Common cofactors include ATP, coenzyme A, flavin adenine dinucleotide (FAD), NAD+/NADH, and heme. Polynucleotides encoding cofactor transport polypeptides and/or cofactor synthesizing polypeptides can be introduced into host cells, and such polypeptides can be constitutively expressed, or inducibly coexpressed with the gene products to be produced by methods of the disclosure.

Glycosylation of polypeptide gene products. Host cells can have alterations in their ability to glycosylate polypeptides. For example, eukaryotic host cells can have eliminated or reduced gene function in glycosyltransferase and/or oligo saccharyltransferase genes, impairing the normal eukaryotic glycosylation of polypeptides to form glycoproteins. Prokaryotic host cells such as E. coli, which do not normally glycosylate polypeptides, can be altered to express a set of eukaryotic and prokaryotic genes that provide a glycosylation function (DeLisa et al., WO 2009/089154A2, 2009 Jul. 16).

Available host cell strains with altered gene functions. To create preferred strains of host cells to be used in the expression systems and methods of the disclosure, it is useful to start with a strain that already comprises desired genetic alterations (Table A; International Publication No. WO 2017/106583).

TABLE A
Exemplary host cell strains

| Strain | Genotype | Source |
|---|---|---|
| E, coli TOP10 | F- mcrA Δ(mrr-hsdRMS-mcrBC) | Invitrogen Life |
| | Φ80lacZ ΔM15 Δlacx74 recA1 araD139 | Technologies Catalog Nos. |
| | Δ(ara-leu)7697 galU galK rpsL (Str^{R}) | C404-10, C4040-03, C4040- |
| | endA1 nupG λ | 06, C4040-50, and C4040-52 |
| E, coli | Δ(ara-leu)7697 Δlacx74 ΔphoA PvuII | Merck (EMD Millipore |
| Origami ™ 2 | phoR araD139 ahpC gale galK rpsL | Chemicals) Catalog No. |
| | F′[lac⁺ lacI^{q} pro] gor522::Tn10 trxB | 71344 |
| | (Str^{R}, Tet^{R}) | |
| E, coli SHuffle ® | fhuA2 [lon] ompT ahpC gal | New England Biolabs |
| Express | λatt::pNEB3-r1-cDsbc (Spec, lacI) | Catalog No. C3028H |
| | ΔtrxB sulA11 R(mcr-73::miniTn10- | |
| | Tet^{S})2 [dcm] R(zgb-210::Tn10-Tet^{S}) | |
| | endA1 Δgor Δ(mcrC-mrr)114::IS10 | |

### Expression Constructs

In some embodiments, a prokaryotic cell described herein comprises one or more expression constructs may optionally include one or more inducible promoters to express an antigen binding protein of interest.

The term “expression construct” as used herein refers to polynucleotides designed for the expression of one or more antigen binding proteins of interest, and thus are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more antigen binding proteins. One or more expression constructs can be integrated into a host cell chromosome or be maintained on an extrachromosomal polynucleotide such as a plasmid or artificial chromosome. The following are descriptions of particular types of polynucleotide sequences that can be used in expression constructs for the expression or coexpression of gene products, including fusion proteins as described herein.

Origins of replication. Expression constructs must comprise an origin of replication, also called a replicon, in order to be maintained within the host cell as independently replicating polynucleotides. Different replicons that use the same mechanism for replication cannot be maintained together in a single host cell through repeated cell divisions. As a result, plasmids can be categorized into incompatibility groups depending on the origin of replication that they contain, as shown in Table 2 of International Publication No. WO 2016/205570. Origins of replication can be selected for use in expression constructs on the basis of incompatibility group, copy number, and/or host range, among other criteria. As described above, if two or more different expression constructs are to be used in the same host cell for the coexpression of multiple gene products, it is best if the different expression constructs contain origins of replication from different incompatibility groups: a pMBI replicon in one expression construct and a pl5A replicon in another, for example. The average number of copies of an expression construct in the cell, relative to the number of host chromosome molecules, is determined by the origin of replication contained in that expression construct. Copy number can range from a few copies per cell to several hundred (Table 2 of WO/2016/205570). In some embodiments, different expression constructs are used which comprise inducible promoters that are activated by the same inducer, but which have different origins of replication. By selecting origins of replication that maintain each different expression construct at a certain approximate copy number in the cell, it is possible to adjust the levels of overall production of a gene product expressed from one expression construct, relative to another gene product expressed from a different expression construct. As an example, to coexpress subunits A and B of a multimeric protein, an expression construct is created which comprises the colEl replicon, the am promoter, and a coding sequence for subunit A expressed from the am promoter: ‘colEl-Para-A.

Another expression construct is created comprising the pl 5A replicon, the am promoter, and a coding sequence for subunit B: ‘pl5A-Para-B’. These two expression constructs can be maintained together in the same host cells, and expression of both subunits A and B is induced by the addition of one inducer, arabinose, to the growth medium. If the expression level of subunit A needed to be significantly increased relative to the expression level of subunit B, in order to bring the stoichiometric ratio of the expressed amounts of the two subunits closer to a desired ratio, for example, a new expression construct for subunit A could be created, having a modified pMB 1 replicon as is found in the origin of replication of the pUC9 plasmid (‘pUC9ori’): pUC9ori-Para-A. Expressing subunit A from a high-copy-number expression construct such as pUC9ori-Para-A should increase the amount of subunit A produced relative to expression of subunit B from pl5A-Para-B. In a similar fashion, use of an origin of replication that maintains expression constructs at a lower copy number, such as pSOOI (WO/2016/205570), could reduce the overall level of a gene product expressed from that construct. Selection of an origin of replication can also determine which host cells can maintain an expression construct comprising that replicon. For example, expression constructs comprising the colEl origin of replication have a relatively narrow range of available hosts, species within the Enterobacteriaceae family, while expression constructs comprising the RK2 replicon can be maintained in E. coli, Pseudomonas aeruginosa, Pseudomonas putida, Azotobacter vinelandii, and Alcaligenes eutrophus, and if an expression construct comprises the RK2 replicon and some regulator genes from the RK2 plasmid, it can be maintained in host cells as diverse as Sinorhizobium meliloti, Agrobacterium tumefaciens, Caulobacter crescentus, Acinetobacter calcoaceticus, and Rhodobacter sphaeroides (Kiies and Stahl, Microbiol Rev 1989 December; 53 (4): 491-516).

Similar considerations can be employed to create expression constructs for inducible expression or coexpression in eukaryotic cells. For example, the 2-micron circle plasmid of Saccharomyces cerevisiae is compatible with plasmids from other yeast strains, such as pSRI (ATCC Deposit Nos. 48233 and 66069; Araki et al., J Mol Biol 1985 Mar. 20; 182 (2): 191-203) and pKDI (ATCC Deposit No. 37519; Chen et al, Nucleic Acids Res 1986 Jun. 11; 14 (11): 4471-4481).

In some embodiments, the expression construct comprises a selection gene. A “selection gene”, also termed a selectable marker, encodes a protein necessary for the survival or growth of a host cell in a selective culture medium. Host cells not containing the expression construct comprising the selection gene will not survive in the culture medium. Typical selection genes encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmIR or CmR), kanamycin (KanR), spectinomycin (SpcR), streptomycin (StrR), and tetracycline (TetR). Many of the plasmids in Table 2 of WO/2016/205570 comprise selectable markers, such as pBR322 (AmpR, TetR); pMOB45 (CmR, TetR); pACYCIW (AmpR, KanR); and pGBMI (SpcR, StrR). The native promoter region for a selection gene is usually included, along with the coding sequence for its gene product, as part of a selectable marker portion of an expression construct. Alternatively, the coding sequence for the selection gene can be expressed from a constitutive promoter.

Exemplary selectable markers include, but are not limited to, neomycin phosphotransferase (npt II), hygromycin phosphotransferase (hpt), dihydrofolate reductase (dhfr), zeocin, phleomycin, bleomycin resistance gene (ble), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2), orotidine-5′-phosphate (OMP) decarboxylase (ura3), and orthologs of any of the foregoing.

Inducible promoter. As described herein, there are several different inducible promoters that can be included in expression constructs as part of the inducible coexpression systems of the disclosure. In some embodiments, inducible promoters share at least 80% polynucleotide sequence identity (more preferably, at least 90% identity, and most preferably, at least 95% identity) to at least 30 (more preferably, at least 40, and most preferably, at least 50) contiguous bases of a promoter polynucleotide sequence as defined in Table 1 of International Publication No. WO 2016/205570 by reference to the E. coli K-12 substrain MG1655 genomic sequence, where percent polynucleotide sequence identity is determined using the methods of Example 11 of WO/2016/205570. Under ‘standard’ inducing conditions (see Example 5 of International Publication No. WO 2016/205570), preferred inducible promoters have at least 75% (more preferably, at least 100%, and most preferably, at least 110%) of the strength of the corresponding ‘wild-type’ inducible promoter of E. coli K-12 substrain MG1655, as determined using the quantitative PCR method of De Mey et al. (Example 6 of International Publication No. WO 2016/205570). Within the expression construct, an inducible promoter is placed 5′ to (or ‘upstream of) the coding sequence for the gene product that is to be inducibly expressed, so that the presence of the inducible promoter will direct transcription of the gene product coding sequence in a 5′ to 3′ direction relative to the coding strand of the polynucleotide encoding the gene product.

Ribosome binding site. For polypeptide gene products, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. Preferably, the region of the expression construct that corresponds to the 5′ UT comprises a polynucleotide sequence similar to the consensus ribosome binding site (RBS, also called the Shine-Dalgarno sequence) that is found in the species of the host cell. In prokaryotes (archaea and bacteria), the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In expression constructs, the RBS sequence is preferably at least 55% identical to the AGGAGGU consensus sequence, more preferably at least 70% identical, and most preferably at least 85% identical, and is separated from the initiation codon by 5 to 10 intervening nucleotides, more preferably by 6 to 9 intervening nucleotides, and most preferably by 6 or 7 intervening nucleotides. The ability of a given RBS to produce a desirable translation initiation rate can be calculated at the website salis.psu.edu/software/RBSLibraryCalculatorSearchMode, using the RBS Calculator; the same tool can be used to optimize a synthetic RBS for a translation rate across a 100,000+ fold range (Salis, Methods Enzymol 2011; 498:19-42).

Multiple cloning site. A multiple cloning site (MCS), also called a polylinker, is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS. Examples of MCS sequences are those in the pBAD series of expression vectors, including pBAD18, pBAD18-Cm, pBAD18-Kan, pBAD24, pBAD28, pBAD30, and pBAD33 (Guzman et al., J Bacteriol 1995 July; 177 (14): 4121-4130); or those in the pPRO series of expression vectors derived from the pBAD vectors, such as pPR018, pPR018-Cm, pPR018-Kan, pPR024, pPRO30, and pPR033 (U.S. Pat. No. 8,178,338 B2; May 15, 2012; Keasling, Jay). A multiple cloning site can be used in the creation of an expression construct: by placing a multiple cloning site 3′ to (or downstream of) a promoter sequence, the MCS can be used to insert the coding sequence for a gene product to be expressed or coexpressed into the construct, in the proper location relative to the promoter so that transcription of the coding sequence will occur. Depending on which restriction enzymes are used to cut within the MCS, there may be some part of the MCS sequence remaining within the expression construct after the coding sequence or other polynucleotide sequence is inserted into the expression construct. Any remaining MCS sequence can be upstream or, or downstream of, or on both sides of the inserted sequence. A ribosome binding site can be placed upstream of the MCS, preferably immediately adjacent to or separated from the MCS by only a few nucleotides, in which case the RBS would be upstream of any coding sequence inserted into the MCS. Another alternative is to include a ribosome binding site within the MCS, in which case the choice of restriction enzymes used to cut within the MCS will determine whether the RBS is retained, and in what relation to, the inserted sequences. A further alternative is to include a RBS within the polynucleotide sequence that is to be inserted into the expression construct at the MCS, preferably in the proper relation to any coding sequences to stimulate initiation of translation from the transcribed messenger RNA.

Expression from constitutive promoters. Expression constructs of the disclosure can also comprise coding sequences that are expressed from constitutive promoters. Unlike inducible promoters, constitutive promoters initiate continual gene product production under most growth conditions. One example of a constitutive promoter is that of the Tn3 bla gene, which encodes beta-lactamase and is responsible for the ampicillin-resistance (AmpR) phenotype conferred on the host cell by many plasmids, including pBR322 (ATCC 31344), pACYCIW (ATCC 37031), and pBAD24 (ATCC 87399). Another constitutive promoter that can be used in expression constructs is the promoter for the E. coli lipoprotein gene, Ipp, which is located at positions 1755731-1755406 (plus strand) in E. coli K-12 substrain MG1655 (Inouye and Inouye, Nucleic Acids Res 1985 May 10; 13 (9): 3101-3110). A further example of a constitutive promoter that has been used for heterologous gene expression in E. coli is the trpLEDCBA promoter, located at positions 1321169-1321133 (minus strand) in E. coli K-12 substrain MG1655 (Windass et al., Nucleic Acids Res 1982 Nov. 11; 10 (21): 6639-6657). Constitutive promoters can be used in expression constructs for the expression of selectable markers, as described herein, and also for the constitutive expression of other gene products useful for the coexpression of the desired product. For example, transcriptional regulators of the inducible promoters, such as AraC, PrpR, RhaR, and XyIR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct. Gene products useful for the production of coexpressed gene products, and the resulting desired product, also include chaperone proteins, cofactor transporters, etc.

Signal Peptides. Polypeptide gene products expressed or coexpressed by the methods of the disclosure can contain signal peptides or lack them, depending on whether it is desirable for such gene products to be exported from the host cell cytoplasm into the periplasm, or to be retained in the cytoplasm, respectively. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides can be characterized functionally by the ability to direct transport of a polypeptide, either co-translationally or post-translationally, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm, using a method such as described in Example 12 of International Publication No. WO 2016/205570.

Examples of inducible promoters and related genes are, unless otherwise specified, from Escherichia coli (E. coli) strain MG1655 (American Type Culture Collection deposit ATCC 700926), which is a substrain of E. coli K-12 (American Type Culture Collection deposit ATCC 10798). Table 1 of International Publication No. WO 2016/205570 lists the genomic locations, in E. coli MG1655, of the nucleotide sequences for these examples of inducible promoters and related genes. Nucleotide and other genetic sequences, referenced by genomic location as in Table 1 of International Publication No. WO 2016/205570, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose—araBAD, araC, arciE, and araFGH—but the terms ‘arabinose promoter’ and ‘ara promoter’ are typically used to designate the araBAD promoter. Several additional terms have been used to indicate the E. coli araBAD promoter, such as Para, ParaB, ParaBAD, and PBAD—The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from PBAD, but in the presence of arabinose, the AraC protein, which alters its conformation upon binding arabinose, becomes a positive regulatory element that allows transcription from PBAD—The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY (AIWC) and the LacY (AIWV) proteins, having a cysteine or a valine amino acid instead of alanine at position 177, respectively (Morgan-Kiss et al., Proc Natl Acad Sci USA 2002 May 28; 99 (11): 7373-7377). In order to achieve homogenous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY (A177C) or LacY (A177V), will not lead to the ‘all or none’ induction phenomenon when the arabinose-inducible promoter is induced by the presence of arabinose. Because the LacY (A177C) protein appears to be more effective in transporting arabinose into the cell, use of polynucleotides encoding the LacY (A177C) protein is preferred to the use of polynucleotides encoding the LacY (A177V) protein.

Propionate promoter. The ‘propionate promoter’ or ‘prp promoter’ is the promoter for the E. coli prpBCDE operon, and is also called PPOB. Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription from the prp promoter when the PrpR protein binds 2-methylcitrate (‘2-MC’). Propionate (also called propanoate) is the ion, CH₃CH₂COO—, of propionic acid (or ‘propanoic acid’), and is the smallest of the ‘fatty’ acids having the general formula H(CH₂)ₙCOOH that shares certain properties of this class of molecules: producing an oily layer when salted out of water and having a soapy potassium salt. Commercially available propionate is generally sold as a monovalent cation salt of propionic acid, such as sodium propionate (CH₃CH₂COONa), or as a divalent cation salt, such as calcium propionate (Ca(CH3CH2COO) 2). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolism of 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-yg/DGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfI, introduced into E. coli BL21 (DE3) to create strain JSB (Lee and Keasling, “A propionate-inducible expression system for enteric bacteria”, Appl Environ Microbiol 2005 November; 71 (11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfI also apparently affects ygfI, which encodes a putative LysR-family transcriptional regulator of unknown function. The genes sbm-yg/DGH are transcribed as one operon, and ygfI is transcribed from the opposite strand. The 3′ ends of the ygfti and ygfI coding sequences overlap by a few base pairs, so a deletion that takes out all of the sbm-yg/DGH operon apparently takes out ygfI coding function as well. Eliminating or reducing the function of a subset of the sbm-ygfDGH gene products, such as YgfG (also called ScpB, methylmalonyl-CoA decarboxylase), or deleting the majority of the sbm-yg/DGH (or scpA-argK-scpBC) operon while leaving enough of the 3′ end of the ygfIi (or scpC) gene so that the expression of ygfI is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter’ or ‘rha promoter’, or PrhaSR, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al, J Bacteriol 2010 January; 192 (1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxy acetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins RmIA, RmIB, RmIC, and RmID (also called RfbA, RfbB, RfbC, and RfbD, respectively) encoded by the rmIBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the RmIA, RmIB, RmIC, and

RmID proteins. L-rhamnose is transported into the cell by RhaT, the rhamnose permease or L-rhamnose: proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or PxyiA, means the promoter for the E. coli xylAB operon. The xylose promoter region is similar in organization to other inducible promoters in that the xylAB operon and the xyIFGHR operon are both expressed from adjacent xylose-inducible promoters in opposite directions on the E. coli chromosome (Song and Park, J Bacteriol. 1997 November; 179 (22): 7025-7032). The transcriptional regulator of both the PxyiA and PxyiF promoters is XyIR, which activates expression of these promoters in the presence of xylose. The xyIR gene is expressed either as part of the xyIFGHR operon or from its own weak promoter, which is not inducible by xylose, located between the xyIH and xyIR protein-coding sequences. D-xylose is catabolized by XyIA (D-xylose isomerase), which converts D-xylose to D-xylulose, which is then phosphorylated by XyIB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XylA, or optionally of both XylA and XyIB. The xyIFGHR operon encodes XyIF, XyIG, and XyIH, the subunits of an ABC super-family high-affinity D-xylose transporter. The xylE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xyIFGHR operon could be altered so that the xyIFGH coding sequences are deleted, leaving XyIR as the only active protein expressed from the xylose-inducible PxyiF promoter, and with the xylE coding sequence expressed from a constitutive promoter rather than its native promoter. As another example, the xyIR coding sequence is expressed from the PxyiA or the promoter in an expression construct, while either the xyIFGHR operon is deleted and xylE is constitutively expressed, or alternatively an xyIFGH operon (lacking the xyIR coding sequence since that is present in an expression construct) is expressed from a constitutive promoter and the xyIE coding sequence is deleted or altered so that it does not produce an active protein.

Lactose promoter. The term ‘lactose promoter’ refers to the lactose-inducible promoter for the lacZYA operon, a promoter which is also called lacZpl; this lactose promoter is located at ca. 365603-365568 (minus strand, with the NA polymerase binding (‘-35’) site at ca. 365603-365598, the Pribnow box (‘-10’) at 365579-365573, and a transcription initiation site at 365567) in the genomic sequence of the E. coli K-12 substrain MG1655 (NCBI Reference Sequence NC 000913.2, II-JAN-2012). In some embodiments, inducible coexpression systems of the disclosure can comprise a lactose-inducible promoter such as the lacZYA promoter. In other embodiments, the inducible coexpression systems of the disclosure comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘-10’) at 401695-401701 (Kikuchi et al., Nucleic Acids Res 1981 Nov. 11; 9 (21): 5671-5678)) in the genomic sequence of the E. coli K-12 substrain MG1655 (NCBI Reference Sequence NC 000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417,142-417,831 and the PhoR coding sequence at 417,889-419,184) in the genomic sequence of the E. coli K-12 substrain MG1655 (NCBI Reference Sequence NC 000913.3, 16 Dec. 2014). The phoA promoter differs from the inducible promoters described above in that it is induced by the lack of a substance-intracellular phosphate-rather than by the addition of an inducer. For this reason the phoA promoter is generally used to direct transcription of gene products that are to be produced at a stage when the host cells are depleted for phosphate, such as the later stages of fermentation. In some embodiments, inducible coexpression systems of the disclosure can comprise a phoA promoter. In other embodiments, the inducible coexpression systems of the disclosure comprise one or more inducible promoters that are not phoA promoters.

As described herein, it may be advantageous or desirable to remove (e.g., by way of an inducible or constitutive “curing” mechanism) an expression construct described herein, e.g., if the cell line harboring the expression construct is or will be used for commercial purposes. Thus, in some embodiments, the expression construct may comprise a “kill switch.” For example, in embodiment, the expression construct includes a temperature-sensitive origin of replication. Additional curing methods are known in the art and include using detergents and intercalating agents, drugs and antibiotics (Buckner, M. M. C., et al., FEMS Microbiology Reviews, fuy031,42, 2018, 781-804).

All patents and other publications identified are expressly incorporated herein by reference in their entirety or in relevant part, as would be apparent from the context of the citation, for the purpose of describing and disclosing, for example, the methodologies described in such publications that might be used in connection with information described herein.

The following examples are given merely to illustrate the present invention and not in any way to limit its scope.

### EXAMPLES

### Materials and Methods

### Sample Preparation: 200 μL Cell Cultures

200 μL er well of cell cultures were transferred into a new 96-Well Sample plate. The 96-Well Sample containing the cell cultures at was centrifuged at 3500×G for 10 minutes at 4° C. The supernatant was discarded, and the cell pellets remained in the plate.

### Sample Preparation: 200 μL Cell Pellets

Obtain a 96-Well plate or plates containing 200 μL cell pellets per well. Cell Lysis Buffer was performed by mixing BugBuster® Protein Extraction Reagent, Protease Inhibitor Cocktail, and Benzonase Nuclease. For a single 96-Well plate, 6.25 mL of BugBuster® Protein Extraction Reagent, 62.5 L of Protease Inhibitor Cocktail, and 5 μL of Benzonase Nuclease were added into a 15 mL Conical-Bottom Centrifuge Tube. The tube was vortexed to mix the Cell Lysis Buffer thoroughly.

The 200 μL cell pellets were resuspended by adding 50 μL of Cell Lysis Buffer into each well. The resuspended samples in the 96-Well plate(s) (covered with 96-Well Plate Adhesive Sealing Film) were mixed for 20 minutes at 37° C. in an incubator shaker.

After 20 minutes, the 96-Well plate(s) were removed from the shaker and 170 μL of 1×HBSTE ((Hepes-buffered saline with 0.05% Tween20 and 3 mM EDTA) buffer was added into each well. The 96-Well containing cell lysates at were centrifuged at 3500×G for 5 minutes. After centrifugation, the supernatant was transferred into 96-Well Filter Plate(s). The plate containing the pellets was discarded.

The mixture was filtered using the Pall or MultiScreen® Vacuum Manifold and a vacuum filtration system.

### Example 1—Direct Antigen Binding Analysis in Bacterial Production Cell Line

Individual engineered E. coli B strains colonies expressing Trastuzumab Fab variants were inoculated in LB media in 96-well deep blocks (Labcon) and grown at 30° C. for 24 hrs to create seed cultures for inducing expression. Seed cultures were then inoculated in IBM media contain inducers and supplements in 96-well deep block and additionally grown at 30° C. for 24 hrs. Post induction samples were transferred to 96-well plates (Greiner Bio-One), pelleted and lysed in 50 μL lysis buffer (BugBuster® protein extraction reagent containing X Benzonase Nuclease and X Protease inhibitor cocktail). Plates were incubated for 15-20 mins at 30° C. then centrifuged to remove insoluble debris. After lysis samples were adjusted with 170 μL SPR running buffer (1×HBSTE, 0.5 mg/MI BSA) to a final volume of 230 μL and filtered into 96-well plates. Lysed samples were then transferred from 96-well plates to 384-well plates for high-throughput SPR using a Hamilton STAR automated liquid handler.

High-throughput surface plasmon resonance (SPR) was conducted on a microfluidic Carterra LSA SPR instrument using SPR running buffer (1×HBSTE, 0.5 mg/Ml BSA). Carterra LSa SAD200M chips were pre-functionalized with 20 ug/ml CaptureSelect™ Human Fab-kappa Kinetics Biotin Conjugate (ThermoFisher) for 10 mins. Lysed samples in 384-well blocks were immobilized onto chip surface for 10 minutes followed 1 min washout step to return to baseline. Antigen binding was conducted using nonregenration kinetics methods with a 5-minute association phase followed by a 15-minute dissociation phase. 6 concentrations of Her2 extracellular domain antigen (ACRO Biosystems) were prepared in a 3-fold serial dilution starting at a concentration of 500 nM. For antigen injections, 6 leading blanks were introduced to create a consistent baseline prior to monitoring antigen binding kinetics. Binding data were corrected and fitted using the kinetics software that accompanies Carterra LSA. Data were fitted to a 1:1 model and ka, kd and Kd values extrapolated from nonlinear regression fits.

### Example 2—Comparison of Stability of Various Antibody Capture Methods

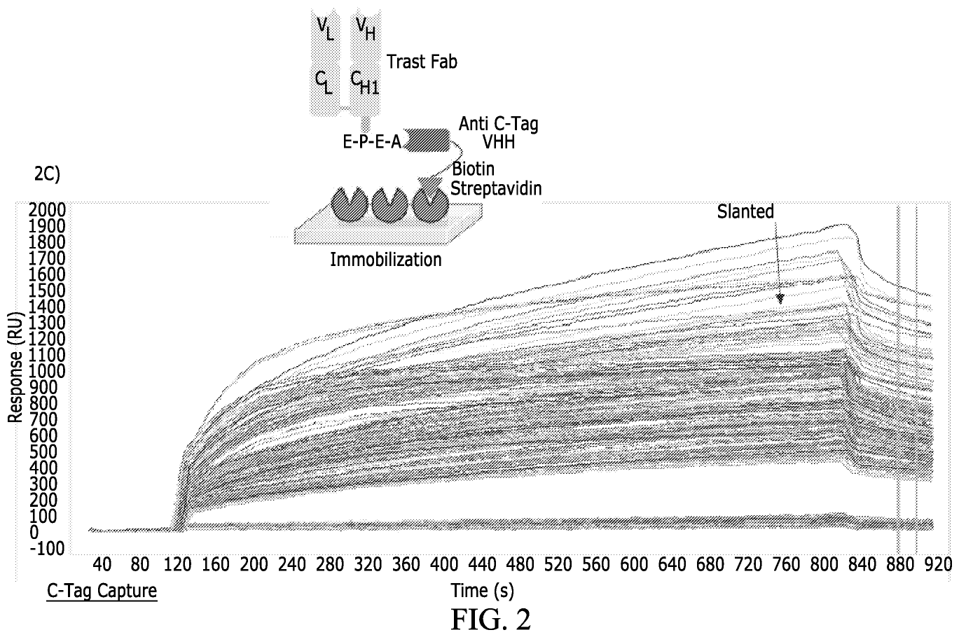

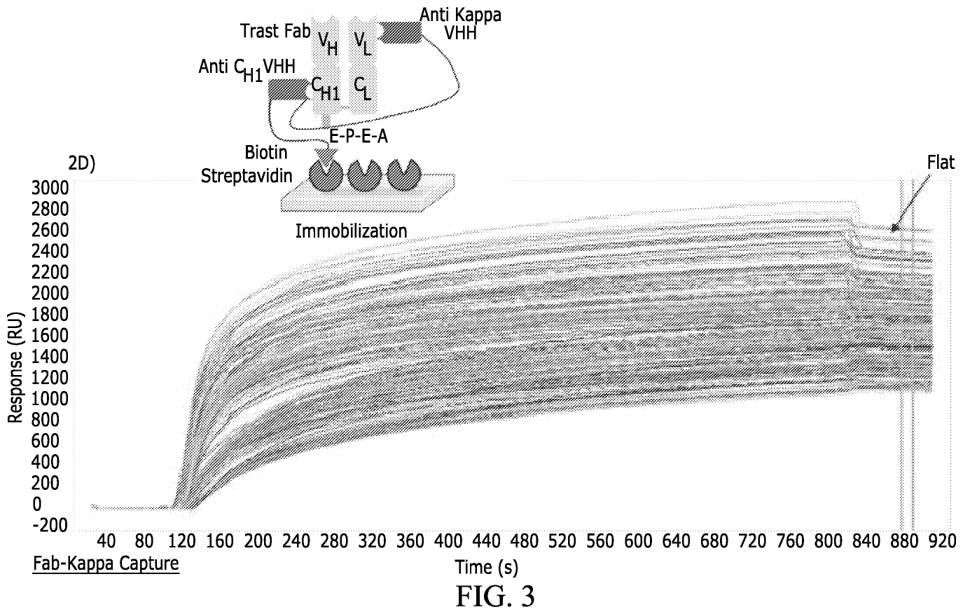

In this experiment, five standards of trastuzumab Fab with Kds ranging from 2 nM-100 nM were expressed in bacteria and a lysate was prepared as described in Example 1. Samples in lysate were loaded onto Carterra chip contain Fab Kappa capture reagent as shown in FIG. 2A or C-tag capture reagent shown in FIG. 2B. FIGS. 2C and 2D show a comparison of the immobilization efficiency of both methods. Fab Kappa method is stable at washout baseline step when samples are completely loaded, while C-tag method shows a clear sloping effect as washout which indicates unstable capture and time-dependent dissociation. C-tag method is not compatible with the Carterra LSA instrument and high throughput kinetics because of a time-dependent loss of signal and antigen binding is not quantifiable. With Fab Kappa Method calculated dissociation constants match published values for 5 standards used in this experiment. The data provided herein confirms that Fab Kappa method of antibody capture is the most efficient for direct antigen binding from lysate.

### Example 3—Validation of the Accuracy Binding Kinetic Parameters

The following experiment describes how variants of Trastuzumab Fab fragment in lysate were analyzed for Her2 binding on Carterra LSA SPR. Kinetics of Fab-Her2 biding from lysate were compared to binding of purified variants by SPR.

Purified variants were obtained from lysate prepared in Example 1 using magnetic beads coated with protein A to bind antibodies and remove unwanted endogenous E. coli proteins. Beads were incubated in cell lysate and then separated using magnetic blocks. Lysate was removed by pipetting and beads washed 5 times with excess 1×PBS (phosphate-buffered saline). Purified proteins were eluted from beads using 10 mM glycine pH3.0 and neutralized with 1M Tris pH 9.0 stock solution to a final concentration of 50 mM. Purified samples were ran on Carterra LSA at a concentration of 20 μg/mL and antigen binding conducted as described in Example 1. For BLI experiments variants were diluted into Gator© BLI K© buffer to 20 μg/mL and immobilized onto streptavidin probes pre-functionalized with Fab Kappa capture reagent. BLI experiments were conducted with same time parameters as Carterra LSA SPR.

A strong correlation was observed between kinetic parameters from lysate and purified samples on SPR and BLI demonstrating the accuracy and sensitivity of Lysate-based antibody binding analysis of the present disclosure.

### Example 4—Validation of the Sensitivity and Limit of Detection of Antibody Binding Kinetics

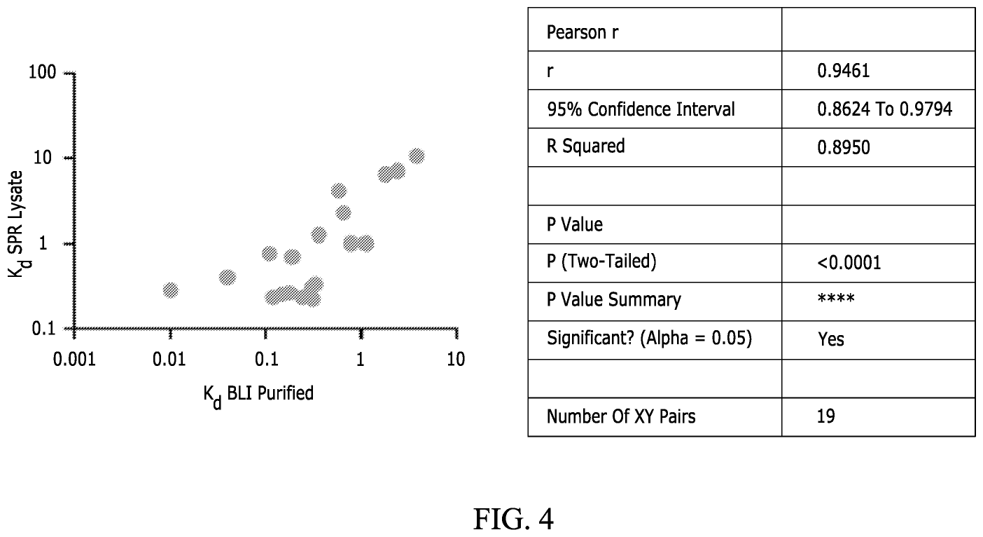

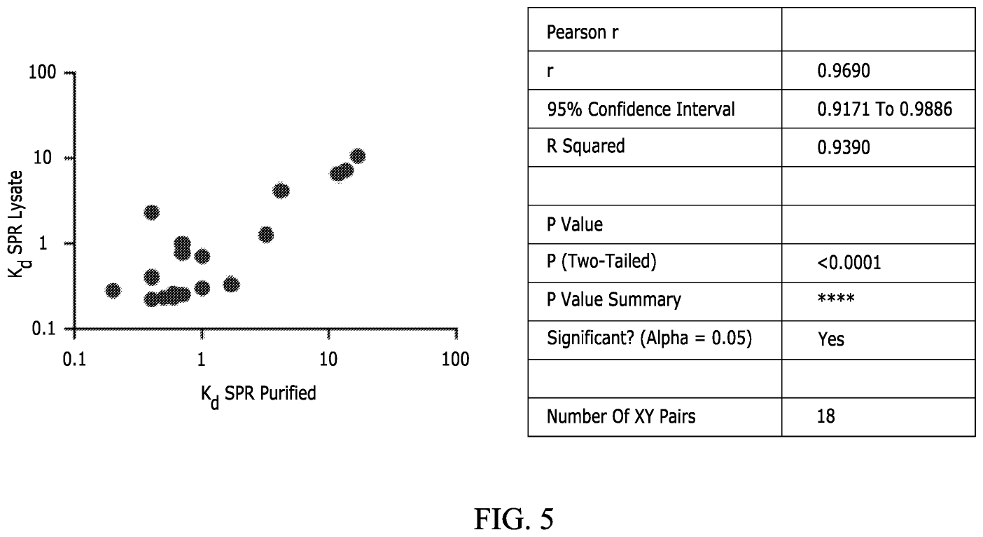

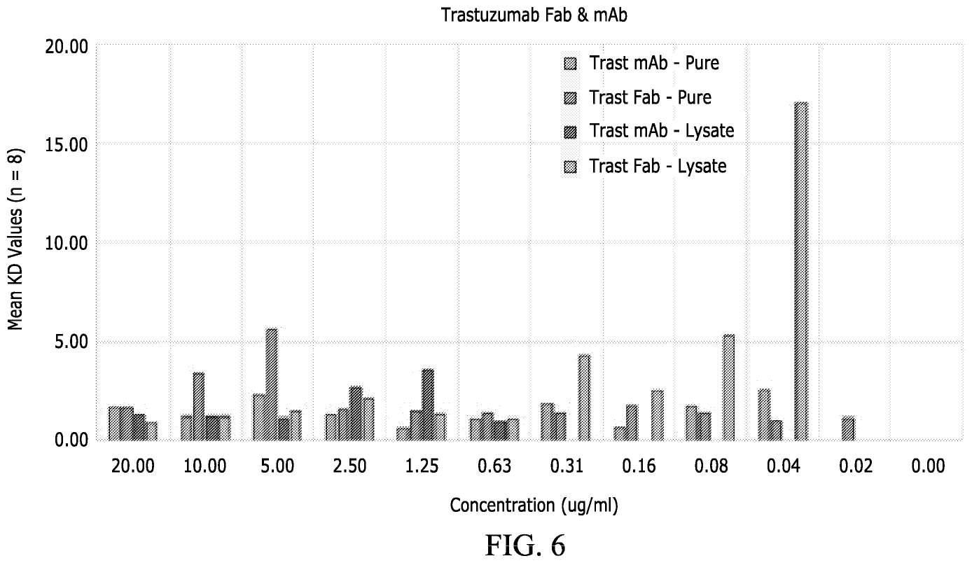

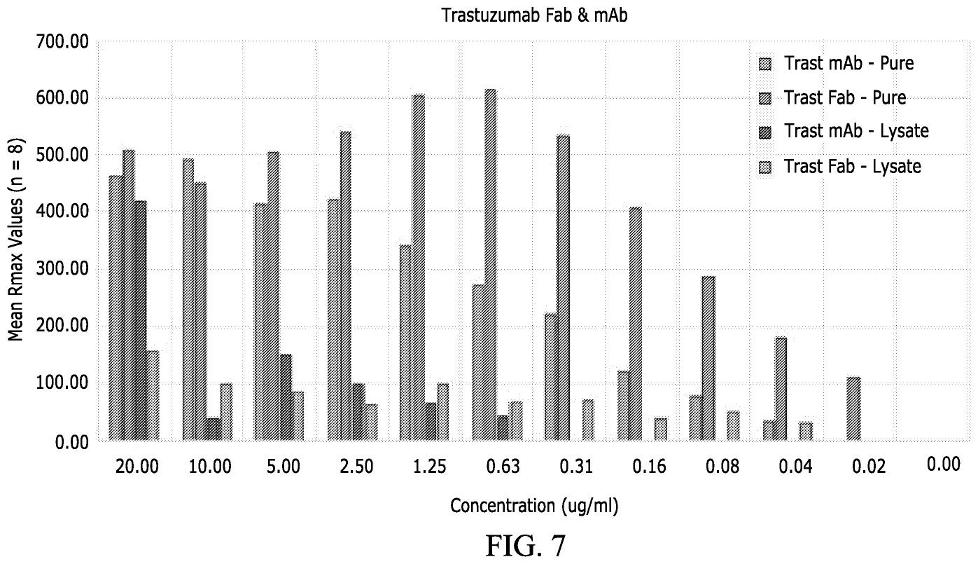

Samples of Purified protein were prepared in a 2-fold serial dilution in lysate or in 1×HBSTE. A series of 12 dilutions were prepared that ranged from 20 μg/mL to 0.02 ug/mL and immobilized on Fab Kappa capture reagent. Binding Kinetics were carried out as described in Example 1. Results show that binding kinetics and Rmax signal is still accurate as low as 0.63 μg/mL antibody immobilization concentration in lysate. See FIG. 4. The data provided herein demonstrates that the methods described herein are sensitive enough to capture low expressing antibody variants and produce accurate antigen-binding kinetics.

What is claimed is:

1. A method of determining affinity of an antigen binding protein binding to a target, the method comprising:
a) expressing an antigen binding protein in a prokaryotic host cell in a multi-well container,
b) lysing the prokaryotic cell to produce a prokaryotic cell lysate;
c) affixing the prokaryotic cell lysate to a substrate; and
d) determining the affinity of the antigen binding protein to the target.

2. The method of claim 1, wherein the prokaryotic host cell comprises one or more or all of:
(a) an alteration of gene function of at least one gene encoding a transporter protein for an inducer of at least one inducible promoter;
(b) a reduced level of gene function of at least one gene encoding a protein that metabolizes an inducer of at least one inducible promoter;
(c) a reduced level of gene function of at least one gene encoding a protein involved in biosynthesis of an inducer of at least one inducible promoter;
(d) an altered gene function of a gene that affects the reduction/oxidation environment of the host cell cytoplasm;
(e) a reduced level of gene function of a gene that encodes a reductase;
(f) at least one expression construct encoding at least one disulfide bond isomerase protein;
(g) at least one polynucleotide encoding a form of DsbC lacking a signal peptide;
and/or
(h) at least one polynucleotide encoding Ervlp.

3. The method of claim 1 or claim 2, wherein the prokaryotic host cell is derived from an Enterobacterial species.

4. The method of claim 3, wherein host cell is Escherichia coli.

5. The method of any one of claims 1-4, wherein the affinity of the antigen binding protein to the target is determined by a high-throughput screening method.

6. The method of claim 5, wherein the high-throughput screening method is surface plasmon resonation (SPR), or Bio-Layer Interferometry (BLI).

7. The method of any one of claims 1-6, wherein the substrate is kappa capture, lambda capture, FLAG or HIS tag capture, Fc capture, Protein A, Protein G, Protein L, or biotin/streptavidin.

8. The method of any one of claims 1-7, wherein the capture agent is kappa capture.

9. The method of anyone of claims 1-8, wherein the antigen binding protein has a K_{D} between 10 PM and 1 μM for the target.

10. The method of any one of claims 1-9, wherein the antigen binding protein is a multi-chain protein.

11. The method of any one of claims 1-10, wherein the antigen binding protein is an antibody.

12. The method of any one of claims 1-10, wherein the antigen binding protein is an antigen binding fragment of an antibody.

13. A method of determining affinity of a multi-chain protein binding to a target, the method comprising:
a) expressing a multi-chain protein in a prokaryotic host cell,
b) lysing the prokaryotic cell to produce a prokaryotic cell lysate;
c) affixing the prokaryotic cell lysate to a substrate; and
d) determining the affinity of the multi-chain protein to the target.

14. The method of claim 13, wherein the prokaryotic host cell comprises one or more or all of:
(a) an alteration of gene function of at least one gene encoding a transporter protein for an inducer of at least one inducible promoter;
(b) a reduced level of gene function of at least one gene encoding a protein that metabolizes an inducer of at least one inducible promoter;
(c) a reduced level of gene function of at least one gene encoding a protein involved in biosynthesis of an inducer of at least one inducible promoter;
(d) an altered gene function of a gene that affects the reduction/oxidation environment of the host cell cytoplasm;
(e) a reduced level of gene function of a gene that encodes a reductase;
(f) at least one expression construct encoding at least one disulfide bond isomerase protein;
(g) at least one polynucleotide encoding a form of DsbC lacking a signal peptide; and/or
(h) at least one polynucleotide encoding Ervlp.

15. The method of claim 13 or claim 14, wherein the prokaryotic host cell is derived from an Enterobacterial species.

16. The method of claim 15, wherein host cell is Escherichia coli.

17. The method of any one of claims 13-16, wherein the expressing step is performed in a multi-well container.

18. The method of any one of claims 13-17, wherein the affinity of the multi-chain protein to the target is determined by a high-throughput screening method.

19. The method of claim 19, wherein the high-throughput screening method is surface plasmon resonation (SPR), or Bio-Layer Interferometry (BLI).

20. The method of any one of claims 14-20, wherein the substrate is kappa capture, lambda capture, FLAG or HIS tag capture, Fc capture, Protein A, Protein G, Protein L, or biotin/streptavidin.

21. The method of claim 20, wherein the capture agent is kappa capture.

22. The method of anyone of claims 13-21, wherein the multi-chain protein has a K_{D} between 10 PM and 1 μM for the target.

23. The method of any one of claims 13-22, wherein the antigen binding protein is an antibody.

24. The method of any one of claims 13-23, wherein the antibody has two heavy chains and two light chains.
